(19) United States
(12) Patent Application Publication
(10) Pub. No.: US 2024/0058292 A1
(43) Pub. Date: Feb. 22, 2024

(54) PHARMACEUTICAL COMPOSITION FOR TREATING CANCER, AND PREPARATION METHOD AND USE THEREOF

(71) Applicant: Yaqin DENG, Liaoyang (CN)

(72) Inventor: Yaqin DENG, Liaoyang (CN)

(21) Appl. No.: 18/259,402

(22) PCT Filed: Dec. 31, 2021
(86) PCT No.: PCT/CN2021/143696

§ 371 Date: Jun. 27, 2023

(30) Foreign Application Priority Data
Dec. 31, 2020 (CN) 202011618811.X

(51) Int. Cl. A61K 31/22; A61K 31/045; A61K 31/10; A61K 31/121; A61P 35/00

(52) U.S. Cl. CPC A61K 31/22; A61K 31/045; A61K 31/10; A61K 31/121; A61P 35/00

(57) ABSTRACT

Clean version of the Abstract A pharmaceutical composition for treating cancer, and a preparation method and use thereof are provided. The pharmaceutical composition includes an ester compound and dimethylsulfoxide (DMSO), a ketone compound and DMSO, or an alcohol compound and DMSO. The pharmaceutical composition is of a great value for a prevention and a treatment of cancer.

### CROSS REFERENCE TO THE RELATED APPLICATIONS

This application is the national phase entry of International Application No. PCT/CN2021/143696, filed on Dec. 31, 2021, which is based upon and claims priority to Chinese Patent Application No. 202011618811.X, filed on Dec. 31, 2020, the entire contents of which are incorporated herein by reference.

### TECHNICAL FIELD

The present disclosure relates to the technical field of medicine, and specifically relates to a pharmaceutical composition for treating cancer, and a preparation method and use thereof.

### BACKGROUND

The global medical community is almost helpless for treatment of cancer such as malignant tumors, and traditional treatment methods for cancer mainly include surgical therapy, radiotherapy, and chemotherapy. When patients at an early stage of cancer receive a surgical resection treatment, only a very small number of the patients may be cured. Radiotherapy has a weak therapeutic effect. The existing chemotherapeutic drugs have limited therapeutic effects, and can only maintain or prolong a short life. The chemotherapeutic drugs currently used in clinical practice are highly toxic and less effective, and cannot effectively cure malignant tumors. In the late 1990s, targeted therapy and immunotherapy began to emerge. Although the targeted therapy and immunotherapy have specified efficacy, the emergence of drug resistant genes is currently the main obstacle for further improvement of efficacy of targeted therapy and immunotherapy.

Therefore, there is an urgent need for a drug with significant efficacy, little toxic and side effects, and high stability to treat malignant tumors.

### SUMMARY

In order to solve the problems existing in the prior art, the present disclosure provides a pharmaceutical composition for treating or preventing cancer, and a preparation method and use thereof. The pharmaceutical composition includes dimethylsulfoxide (DMSO) and an ester compound. The pharmaceutical composition of the present disclosure is of a great value for prevention and treatment of cancer.

The present disclosure is implemented by the following technical solutions:
In an aspect, the present disclosure provides a pharmaceutical composition for treating or preventing cancer, including an ester compound and DMSO,
where the ester compound is preferably a C₂₋₈ lower ester, more preferably a C₂₋₄ lower ester, and further more preferably ethyl acetate; and
preferably, the pharmaceutical composition includes the ethyl acetate and the DMSO.
Preferably, a volume ratio of the ethyl acetate to the DMSO is 1:1 to 1:200;
preferably, the volume ratio of the ethyl acetate to the DMSO is 1:100 to 1:200;
preferably, the volume ratio of the ethyl acetate to the DMSO is 1:75 to 1:150;
preferably, the volume ratio of the ethyl acetate to the DMSO is 1:100;
preferably, the ethyl acetate and the DMSO are separately diluted with water or mixed and then diluted with water, and then directly administered orally, injected, or sprayed to treat or prevent an animal or human disease; and
preferably, the pharmaceutical composition further includes 4.55 v/v % lactic acid.

The present disclosure also provides a pharmaceutical composition for treating or preventing cancer, including a ketone compound and DMSO,
where the ketone compound is preferably alkanone, more preferably C₃₋₆ alkanone, and further more preferably acetone; and
preferably, the pharmaceutical composition includes the acetone and the DMSO.

Preferably, a volume ratio of the acetone to the DMSO is 1:1 to 1:200;
preferably, the volume ratio of the acetone to the DMSO is 1:100 to 1:200;
preferably, the volume ratio of the acetone to the DMSO is 1:75 to 1:150;
preferably, the acetone and the DMSO are separately diluted with water or mixed and then diluted with water, and then directly administered orally, injected, or sprayed to treat or prevent an animal or human disease; and
preferably, the pharmaceutical composition further includes 4.55 v/v % lactic acid.

The present disclosure also provides a pharmaceutical composition for treating or preventing cancer, including an alcohol compound and DMSO,
where the alcohol compound is preferably Ci-6 alkanol, more preferably C₁₋₄ alkanol, and further more preferably ethanol; and
preferably, the pharmaceutical composition includes the ethanol and the DMSO.

Preferably, a volume ratio of the ethanol to the DMSO is 1:1 to 1:200;
preferably, the volume ratio of the ethanol to the DMSO is 1:100 to 1:200;
preferably, the volume ratio of the ethanol to the DMSO is 1:75 to 1:150; preferably, the ethanol and the DMSO are separately diluted with water or mixed and then diluted with water, and then directly administered orally, injected, or sprayed to treat or prevent an animal or human disease; and
preferably, the pharmaceutical composition further includes 4.55 v/v % lactic acid.

In another aspect, the present disclosure provides a use of the pharmaceutical composition described above in preparation of a drug for preventing and/or treating diseases of cancer, a cancer complication, cerebral edema, diabetes, hypertension, a cardiovascular disease (CVD), lupus erythematosus, pleural effusion, ascites, and gout.

Preferably, an administration route of the pharmaceutical composition includes, but is not limited to, oral administration, intravenous drip, intravenous injection, and transdermal administration.

Preferably, the cancer includes, but is not limited to, glioma, astrocytoma, brain or central nervous system (CNS) cancer, and peripheral nervous system (PNS) cancer, including melanoma, B-cell carcinoma, multiple myeloma, breast cancer, lung cancer, bronchial cancer, colorectal cancer (CRC), prostate cancer, pancreatic cancer, gastric cancer, ovarian cancer, bladder cancer, esophageal cancer, cervical cancer, uterine or endometrial cancer, oral cancer, pharyngeal cancer, liver cancer, kidney cancer, testicular cancer, biliary tract cancer, small intestine cancer or appendiceal cancer, salivary gland cancer, thyroid cancer, adrenal carcinoma, osteosarcoma, chondrosarcoma, blood tissue cancer, adenocarcinoma, inflammatory myofibroblastic tumor (IMT), gastrointestinal stromal tumor (GIST), colon cancer, Hodgkin lymphoma (HL), non-Hodgkin lymphoma (NHL), soft tissue sarcoma (STS), fibrosarcoma, myxosarcoma, liposarcoma, osteogenic sarcoma, chordoma, angiosarcoma, endothelial sarcoma, lymphangiosarcoma, lymphangioendothelial sarcoma, synovioma, mesothelioma, Ewing's sarcoma, leiomyosarcoma, rhabdomyosarcoma, squamous cell carcinoma (SCC), basal cell carcinoma (BCC), sweat gland carcinoma, sebaceous gland carcinoma, papillary carcinoma, papillary adenocarcinoma, medullary carcinoma, renal cell carcinoma (RCC), liver cancer, cholangiocarcinoma, choriocarcinoma, seminoma, embryonal carcinoma, nephroblastoma, bladder cancer, epithelial cancer, medulloblastoma, craniopharyngioma, ependymoma, pinealoma, hemangioblastoma, acoustic neuroma, oligodendroglioma, meningoma, neuroblastoma, retinoblastoma, follicular lymphoma (FL), diffuse large B-cell lymphoma (DLBCL), mantle cell lymphoma (MCL), hepatocellular carcinoma (HCC), thyroid cancer, head and neck cancer, small cell carcinoma, agnogenic myeloid metaplasia (AMM), hypereosinophilic syndrome (HES), chronic eosinophilic leukemia (CEL), neuroendocrine carcinoma (NEC), carcinoid tumor, and metastatic and invasive lesions thereof. The pharmaceutical composition of the present disclosure is also suitable for treatment of diabetes, CVD, lupus erythematosus, pleural effusion, ascites, and gout.

A target of the pharmaceutical composition of the present disclosure is an aldehyde compound attached to a cell membrane; and the pharmaceutical composition anchors an aldehyde compound or an aldehyde protein attached to a cell membrane by allowing an alcohol, ester, ketone, or acid compound to react with the aldehyde compound or the aldehyde protein, and inhibits proliferation, infiltration, or agglomeration of tumor cells by changing a surrounding environment of the tumor cells to suppress growth of a tumor.

The pharmaceutical composition of the present disclosure is a broad-spectrum anticancer drug, and should be clinically administered alone, alternately, or in combination with another drug at different doses specified by a physician with rich experience according to different phases and different symptoms of diseases of patients to allow excellent therapeutic effects.

Compared with the prior art, the pharmaceutical composition of the present disclosure has the following advantages:
(1) The pharmaceutical composition of the present disclosure is obtained by directly mixing a pharmaceutical component with a solvent, which is simple and convenient. However, in an animal experiment, in the pharmaceutical composition, 4.55% lactic acid is added in a specified proportion to allow an improved control effect for a tumor. The pharmaceutical composition is suitable for treatment of various types of cancer.
(2) The pharmaceutical composition of the present disclosure is an accurate tumor inhibitor, and the tumor inhibitor exhibits a pharmacodynamic effect only for malignant tumor cells and does not damage normal tissue cells. Therefore, the pharmaceutical composition of the present disclosure can suppress the proliferation of malignant tumor cells and the infiltration of normal tissue cells by malignant tumor cells without killing and inhibiting normal tissue cells.
(3) Physical and chemical indexes of the solvent used in the pharmaceutical composition of the present disclosure are clear and easy to control, and the pharmacokinetics, toxicity, or the like of the solvent can be easily and clearly determined by professionals and patients.
(4) The pharmaceutical composition of the present disclosure has low toxicity compared with anticancer drugs that have been marketed inside and outside China, and the toxicity may even be negligible; and the pharmaceutical composition has no side effects. Therefore, when administered at an accurate normal dose, the pharmaceutical composition will not cause any harm to the human body.
(5) Unlike existing anti-tumor drugs, the pharmaceutical composition of the present disclosure can effectively inhibit the secondary growth of tumor cells and nodules in other tissues (so-called tumor metastasis) while inhibiting the growth of a solid tumor.
(6) The pharmaceutical composition of the present disclosure can effectively reduce the transmembrane impedance and enhance the cell membrane permeability, such that tissue cells of a patient can timely and effectively utilize glucose, salts, water, or the like to enhance the physical performance of the patient.
(7) The pharmaceutical composition of the present disclosure denatures outer membrane proteins (OMPs) of tumor cells and affects a tumor environment to suppress the massive proliferation of tumor cells and prevent the tumor cells from infiltrating normal tissue cells by. When the pharmaceutical composition makes a proliferation rate of tumor cells lower than a proliferation rate of normal tissue cells and makes the tumor cells stop to infiltrate normal tissue cells, a volume of a tumor is gradually reduced or remains unchanged.

It should be understood that the invention herein is not limited to specific methodologies, experimental schemes, or reagents, which may vary. The discussions and examples provided herein are intended to merely describe specific embodiments, rather than limit the scope of the present disclosure; and the scope of the present disclosure is limited only by the claims.

### BRIEF DESCRIPTION OF THE DRAWINGS

FIG. 1 shows a tumor-suppressing rate of the pharmaceutical composition of the present disclosure;

FIG. 2 is an anatomical atlas of tumors, where experimental results of a solvent control group, a 5-fluorouracil group, a temozolomide (TMZ) group, and high-dose, medium-dose, and low-dose pharmaceutical composition groups are shown sequentially from top to bottom;

FIG. 3 shows tumor volume change trends during administration; and

FIG. 4 shows body weight change trends during administration.

### DETAILED DESCRIPTION OF THE EMBODIMENTS

The experimental materials and sources thereof in the following examples are as follows:

DMSO: (>99.5%), Sinopharm Chemical Reagent Co., Ltd., batch No.:20181010; ethanol: (>95.0%), Sinopharm Chemical Reagent Co., Ltd., batch No.:20200810; acetone: (>99.5%), Sinopharm Chemical Reagent Co., Ltd., batch No.:20140221; and ethyl acetate: (>99.5%), Sinopharm Chemical Reagent Co., Ltd., batch No.:20161026.

### Example 1a Pharmaceutical Composition of the Present Disclosure

Formula: DMSO: 200 mL, and ethyl acetate: 1 mL;
formula: DMSO: 180 mL, and ethyl acetate: 1 mL;
formula: DMSO: 160 mL, and ethyl acetate: 1 mL;
formula: DMSO: 140 mL, and ethyl acetate: 1 mL;
formula: DMSO: 120 mL, and ethyl acetate: 1 mL;
formula: DMSO: 100 mL, and ethyl acetate: 1 mL;
formula: DMSO: 80 mL, and ethyl acetate: 1 mL;
formula: DMSO: 60 mL, and ethyl acetate: 1 mL;
formula: DMSO: 40 mL, and ethyl acetate: 1 mL;
formula: DMSO: 20 mL, and ethyl acetate: 1 mL; and
formula: DMSO: 1 mL, and ethyl acetate: 1 mL.

### Example 2 a Pharmaceutical Composition of the Present Disclosure

Formula: DMSO: 200 mL, and acetone: 1 mL;
formula: DMSO: 180 mL, and acetone: 1 mL;
formula: DMSO: 160 mL, and acetone: 1 mL;
formula: DMSO: 140 mL, and acetone: 1 mL;
formula: DMSO: 120 mL, and acetone: 1 mL;
formula: DMSO: 100 mL, and acetone: 1 mL;
formula: DMSO: 80 mL, and acetone: 1 mL;
formula: DMSO: 60 mL, and acetone: 1 mL;
formula: DMSO: 40 mL, and acetone: 1 mL;
formula: DMSO: 20 mL, and acetone: 1 mL; and
formula: DMSO: 1 mL, and acetone: 1 mL.

### Example 3 a Pharmaceutical Composition of the Present Disclosure

Formula: DMSO: 200 mL, and ethanol: 1 mL;
formula: DMSO: 180 mL, and ethanol: 1 mL;
formula: DMSO: 160 mL, and ethanol: 1 mL;
formula: DMSO: 140 mL, and ethanol: 1 mL;
formula: DMSO: 120 mL, and ethanol: 1 mL;
formula: DMSO: 100 mL, and ethanol: 1 mL;
formula: DMSO: 80 mL, and ethanol: 1 mL;
formula: DMSO: 60 mL, and ethanol: 1 mL;
formula: DMSO: 40 mL, and ethanol: 1 mL;
formula: DMSO: 20 mL, and ethanol: 1 mL; and
formula: DMSO: 1 mL, and ethanol: 1 mL.

### Example 4 Effect of the Pharmaceutical Composition of the Present Disclosure in Treatment of cancer

1. Main instruments and devices

A common laboratory dissection instrument, a biological microscope, an animal body weight-measuring balance, an analytical balance, a water bath, a pipette, a vortex mixer, a biological safety cabinet, a cell culture incubator, or the like.

2. Reagents

A disinfectant, normal saline (NS) for injection, medical alcohol, or the like.

3. Positive drugs

3.1 Positive control 1
Name: Fluorouracil injection
Production unit: Shanghai Xudong Haipu Pharmaceutical Co., Ltd.
Batch No.: FA181206
Package: Glass ampoule
Specification: 10 mL: 0.25 g
Properties: Colorless clear liquid
Storage conditions: It is stored at 15° C. to 25° C. in the dark and should not be frozen.
Expiration date: Dec. 12, 2020

3.2 Positive control 2
Name: TMZ for injection
Production unit: Jiangsu Hengrui Pharmaceuticals Co., Ltd.
Batch No.: 190711AM
Package: Vial
Specification: 100 mg/vial
Properties: White lyophilized lumpy substance
Storage conditions: Closed storage (2° C. to 8° C.)
Expiration date: Jul. 10, 2021

4. Laboratory animals
Animal species: BALB/C-nu/nu mice
Animal grade: SPF grade
Gender and quantity:42 females
Animal age at the start of an experiment: 6 weeks old
Animal body weight at the start of an experiment: 18±2 g
Animal source: Vital River Laboratory Animal Technology Co., Ltd.

5. Quarantine Inspection

Experimental animals were accepted and inspected according to experimental requirements. The adaptation observation was conducted for 5 d, and during the observation, the eyes, ears, nose, mouth, fur, abdomen, vulva, perianal area, limbs, claws, pads, gait, behavior, excretion, food intake, and water drinking of each mouse were observed. Mice with normal quarantine results were selected for the experiment.

6. Grouping

Mice with normal quarantine results were selected and inoculated with tumors, then randomly grouped, and raised separately in labeled cages.

7. Raising conditions

Marine Biomedical Research Institute of Qingdao, experimental animal use license No.:

SYXK (Lu) 2015 0011

Laboratory temperature: 20° C. to 25° C.; humidity: 40% to 70%; number of air changes: 10 to 20 times/h; 12 h light/12 h dark alternating cycles; and raising density: less than 5 mice/cage. A raising environment was created strictly in accordance with relevant standards of the National Standard of the People's Republic of China —Laboratory Animals.

8. Tumor cell line information

Cell source and cultivation conditions: Human brain astroblastoma cell line U-87MG: which came from the Cell Resource Center of the Shanghai Institutes for Biological Sciences (SIBS); medium: MEM medium including 1% penicillin-streptomycin sulfate mixed solution (100 ×) and 10% fetal bovine serum (FBS) (from Australia); cultivation conditions: 5% CO2 and 37° C.; and the cell line was frozen by Marine Biomedical Research Institute of Qingdao.

9. Cell recovery and passage

The cell line U-87MG stored in liquid nitrogen was recovered, inoculated in a corresponding medium including 10% FBS, 100 U/mL penicillin, and 100 1.1 g/mL streptomycin, and cultivated in the cell culture incubator at 37° C. and 5% CO2. The cells were passaged every two days.

10. Tumor inoculation

After the cells were passaged four times, resulting cells were collected through digestion with 0.05% trypsin-EDTA, counted under a microscope, and resuspended with a medium to obtain a cell suspension with 5×10 7 cells/mL; a mouse was routinely disinfected, and 0.2 mL of the cell suspension was subcutaneously inoculated in an axillary region of a right forelimb of the mouse; and a resulting tumor tissue was aseptically taken out, weighed, diluted with a sodium chloride injection according to a mass-to-volume ratio of 1:4, and then ground, then a mouse was routinely disinfected, and 0.2 mL of a resulting suspension was subcutaneously inoculated in an axillary region of a right forelimb of the mouse for passage, where the passage was continuously conducted twice. During this period, the mice were raised normally, and the states and tumor growth conditions of the mice were observed and recorded every week.

When a tumor volume in a tumor-bearing mouse to be passaged was about 1,200 mm 3, a tumor was ground and transplanted by a same method for molding. After the tumor inoculation was completed, a body weight of a mouse was measured, and mice were randomly selected and divided into 6 groups with 7 mice per group. After tumor colonization, the administration was started, which was recorded as Day 1 (D1).

11. Animal grouping and dose

According to body weights, animals were randomly divided into 6 groups, with 7 animals per group. A dose for each group of animals was shown in the table below:

TABLE 1
Administration for each group

| | |
|---|---|
| | Number |
| | of |
| Administration | animals |

| Group | Drug | route | Dose | Frequency | Volume | (n) |
|---|---|---|---|---|---|---|
| Solvent control group | NS | Intragastric | — | Once every day | 0.2 mL | 7 |
| (model group) | | administration | | | | |
| TMZ | TMZ | Intraperitoneal | 25 mg/kg | Once every day | 0.2 mL | 7 |
| | | injection | | | | |
| Fluorouracil | 5-Fluorouracil | Intraperitoneal | 18 mg/kg | Twice a week | 0.2 mL | 7 |
| | | injection | | | | |
| Low-dose inhibitor | Ethyl acetate and | Intragastric | 0.54 mL/kg | Once every day | 0.108 mL | 7 |
| group | DMSO in a volume | administration | (stock solution) | | (diluent) | |
| | ratio of 1:100 | | | | | |
| Medium-dose inhibitor | Ethyl acetate and | Intragastric | 2.7 mL/kg | Once every day | 0.54 mL | 7 |
| group | DMSO in a volume | administration | (stock solution) | | (diluent) | |
| | ratio of 1:100 | | | | | |
| High-dose inhibitor | Ethyl acetate and | Intragastric | 13.5 mL/kg | Twice every day | 2.7 mL | 7 |
| group | DMSO in a volume | administration | (stock solution) | | (diluent) | |
| | ratio of 1:100 | | | | | |

12. Clinical observation

12.1 General clinical observation

Observed animals: All animals

Observation frequency and time: The animals were observed once before each time of administration during the experiment, and administration and observation results were recorded.

Observation content: including, but not limited to, tumor growth, animal mental states, eating and drinking conditions, or the like.

12.2 Body weight

Detected animals: All animals

Detection time: A body weight of an animal was measured and recorded when grouping (namely, before the first administration) (D1), before each subsequent administration, and before euthanasia.

A body weight and relevant information of an animal were measured and recorded if the animal died unexpectedly.

12.3 Efficacy evaluation according to tumor weights

After the experiment was completed, tumor tissues of animals that died unexpectedly and surviving animals that were euthanized each were peeled off and weighed, and a tumor weight difference of each group was calculated to further calculate a tumor-suppressing rate (IRTW) according to the following formula:

IRTW (%)=(W_{model group}−W_{administration group})/W_{model group}×100%.

13. Photo records

After an animal was euthanized, a tumor tissue was peeled off and photographed.

14. Data acquisition and statistical analysis

It was required in this scheme that measured and observed data results were manually recorded in appropriate forms or directly acquired by a computer. The data results were raw data for analytical processing and reporting. The results were expressed as mean±standard deviation (Mean±SD). The t-test was used for comparison between two groups, and both statistical significance and biological significance were considered when the results were analyzed.

15. Results

15.1 General clinical observation

When a high-dose inhibitor group was administered twice every day at a dose of 13.5 mL/kg in total, two mice in the high-dose inhibitor group died on day 3, and the rest mice were crouched, had no mucus outflow from corners of a mouth, nose, or the like, closed eyes, and a reduced body surface temperature (measured by hands), were inactive, and did not eat. A dose of the high-dose inhibitor group was reduced to 6.75 mL/kg, no death occurred until the end of the experiment. Mice in the rest groups were in excellent states.

15.2 Statistics of tumor-suppressing rates

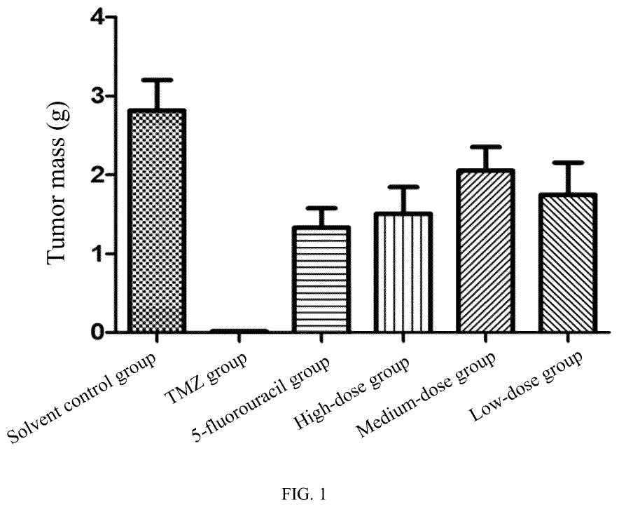

Statistical results of tumor-suppressing rates were shown in FIG. 1 and Table 2.

TABLE 2

| | | | Tumor- |
|---|---|---|---|
| | | Number of | suppress- |
| Group | Tumor weight | samples (n) | ing rate (%) |
| Solvent control group | 2.8155 ± 1.0187 | 7 | — |
| (model group) | | | |
| TMZ | 0.0134 ± 0.0091 | 7 | 99.5% |
| 5-Fluorouracil | 1.3305 ± 0.6525 | 7 | 52.7% |
| Low-dose | 1.7472 ± 1.0790 | 7 | 37.9% |
| inhibitor group | | | |
| Medium-dose | 2.0529 ± 0.7893 | 7 | 27.1% |
| inhibitor group | | | |
| High-dose | 1.5087 ± 0.7484 | 5 | 46.4% |
| inhibitor group | | | |

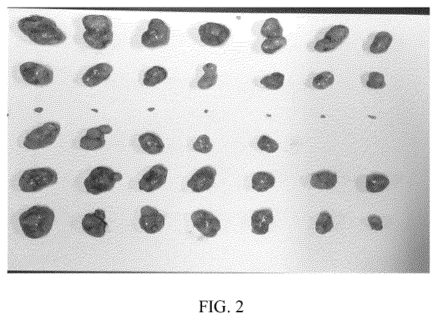

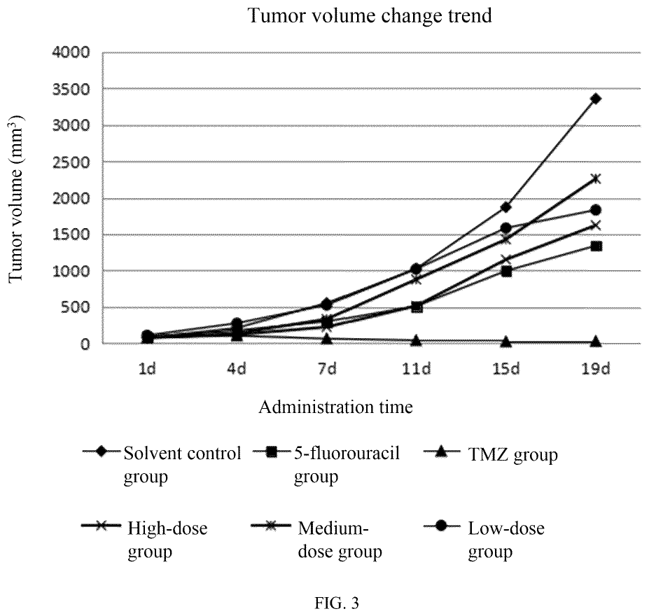

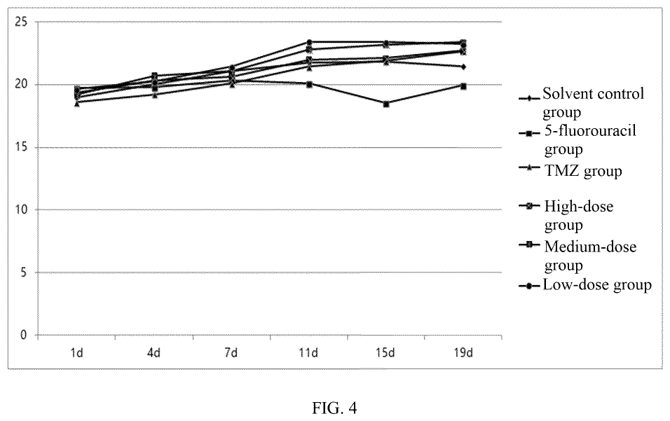

FIG. 2 is an anatomical atlas of tumors, where the solvent control group, the 5-fluorouracil group, the TMZ group, and the high-dose, medium-dose, and low-dose inhibitor groups are shown sequentially from top to bottom. FIG. 3 shows tumor volume change trends during administration. FIG. 4 shows body weight change trends during administration.

It can be seen from the experimental data that, on day 7 of administration after the administration dose is adjusted, an obvious difference begins to occur in a tumor volume; on day 11 to day 15 of administration, tumor-suppressing rates of the high-dose, medium-dose, and low-dose inhibitor groups are dose-dependent; according to final autopsy results, a tumor-suppressing rate of the high-dose inhibitor group was 46.4%; and during administration, except that a body weight of mice in the 5-fluorouracil group is reduced at a late stage of administration, a body weight of mice in other groups normally grows, indicating that, after mice adapt to the drug, the drug has no obvious impact on eating, digestion, and other activities of the mice.

What is claimed is:

1. A pharmaceutical composition for treating or preventing a cancer,
comprising an ester compound and dimethylsulfoxide (DMSO).

2. (canceled)

3. A pharmaceutical composition for treating or preventing a cancer,
comprising a ketone compound and DMSO.

4. (canceled)

5. A pharmaceutical composition for treating or preventing a cancer,
comprising an alcohol compound and DMSO.

6. (canceled)

7. A method of using the pharmaceutical composition according to claim 1 in a preparation of a drug for preventing and/or treating diseases of a cancer, a cancer complication, a cerebral edema, a diabetes, a hypertension, a cardiovascular disease (CVD), a lupus erythematosus, a pleural effusion, an ascites, and a gout.

8. The method according to claim 7, wherein an administration route of the pharmaceutical composition comprises an oral administration, an intravenous drip, an intravenous injection, and a transdermal administration.

9. The method according to claim 7, wherein the cancer comprises a glioma, an astrocytoma, a brain or central nervous system (CNS) cancer, and a peripheral nervous system (PNS) cancer, comprising a melanoma, a B-cell carcinoma, a multiple myeloma, a breast cancer, a lung cancer, a bronchial cancer, a colorectal cancer (CRC), a prostate cancer, a pancreatic cancer, a gastric cancer, an ovarian cancer, a bladder cancer, an esophageal cancer, a cervical cancer, an uterine or endometrial cancer, an oral cancer, a pharyngeal cancer, a liver cancer, a kidney cancer, a testicular cancer, a biliary tract cancer, a small intestine cancer or an appendiceal cancer, a salivary gland cancer, a thyroid cancer, an adrenal carcinoma, an osteosarcoma, a chondrosarcoma, a blood tissue cancer, an adenocarcinoma, an inflammatory myofibroblastic tumor (IMT), a gastrointestinal stromal tumor (GIST), a colon cancer, a Hodgkin lymphoma (HL), a non-Hodgkin lymphoma (NHL), a soft tissue sarcoma (STS), a fibrosarcoma, a myxosarcoma, a liposarcoma, an osteogenic sarcoma, a chordoma, an angiosarcoma, an endothelial sarcoma, a lymphangiosarcoma, a lymphangioendothelial sarcoma, a synovioma, a mesothelioma, a Ewing's sarcoma, a leiomyosarcoma, a rhabdomyosarcoma, a squamous cell carcinoma (SCC), a basal cell carcinoma (BCC), a sweat gland carcinoma, a sebaceous gland carcinoma, a papillary carcinoma, a papillary adenocarcinoma, a medullary carcinoma, a renal cell carcinoma (RCC), a liver cancer, a cholangiocarcinoma, a choriocarcinoma, a seminoma, an embryonal carcinoma, a nephroblastoma, a bladder cancer, an epithelial cancer, a medulloblastoma, a craniopharyngioma, an ependymoma, a pinealoma, a hemangioblastoma, an acoustic neuroma, an oligodendroglioma, a meningoma, a neuroblastoma, a retinoblastoma, a follicular lymphoma (FL), a diffuse large B-cell lymphoma (DLBCL), a mantle cell lymphoma (MCL), a hepatocellular carcinoma (HCC), a thyroid cancer, a head and neck cancer, a small cell carcinoma, an agnogenic myeloid metaplasia (AMM), a hypereosinophilic syndrome (HES), a chronic eosinophilic leukemia (CEL), a neuroendocrine carcinoma (NEC), a carcinoid tumor, and metastatic and invasive lesions thereof.

10. The method according to claim 7, wherein a target of the pharmaceutical composition is an aldehyde compound attached to a cell membrane; and the pharmaceutical composition anchors the aldehyde compound or an aldehyde protein attached to the cell membrane by allowing an alcohol, an ester, ketone, or acid compound to react with the aldehyde compound or the aldehyde protein, and inhibits a proliferation, an infiltration, or an agglomeration of tumor cells by changing a surrounding environment of the tumor cells to suppress a growth of a tumor.

11. A method for treating or preventing a cancer, comprising:
administering the pharmaceutical composition according to claim 1 to a subject in need,
wherein the cancer comprises an astrocytoma, a brain or CNS cancer, and aPNS cancer, comprising a melanoma, a B-cell carcinoma, a multiple myeloma, a breast cancer, a lung cancer, a bronchial cancer, a CRC, a prostate cancer, a pancreatic cancer, &gastric cancer, an ovarian cancer, a bladder cancer, an esophageal cancer, a cervical cancer, an uterine or endometrial cancer, an oral cancer, a pharyngeal cancer, a liver cancer, a kidney cancer, a testicular cancer, a biliary tract cancer, a small intestine cancer or an appendiceal cancer, a salivary gland cancer, a thyroid cancer, an adrenal carcinoma, an osteosarcoma, a chondrosarcoma, a blood tissue cancer, an adenocarcinoma, an IMT a GIST, a colon cancer, an HL, an NHL, an STS, a fibrosarcoma, a myxosarcoma, a liposarcoma, an osteogenic sarcoma, a chordoma, an angiosarcoma, an endothelial sarcoma, a lymphangiosarcoma, a lymphangioendothelial sarcoma, a synovioma, a mesothelioma, an Ewing's sarcoma, a leiomyosarcoma, a rhabdomyosarcoma, an SCC, a BCC, a sweat gland carcinoma, a sebaceous gland carcinoma, a papillary carcinoma, a papillary adenocarcinoma, a medullary carcinoma, an RCC, a liver cancer, a cholangiocarcinoma, a choriocarcinoma, a seminoma, an embryonal carcinoma, a nephroblastoma, a bladder cancer, an epithelial cancer, a medulloblastoma, a craniopharyngioma, an ependymoma, a pinealoma, a hemangioblastoma, an acoustic neuroma, an oligodendroglioma, a meningoma, a neuroblastoma, a retinoblastoma, an FL, a DLBCL, an MCL, an HCC, a thyroid cancer, a head and neck cancer, a small cell carcinoma, an AMM, an HES, a CEL, an NEC, a carcinoid tumor, and metastatic and invasive lesions thereof.

12. The pharmaceutical composition according to claim 1, wherein the ester compound is a C₂₋₈ lower ester.

13. The pharmaceutical composition according to claim 1, wherein the ester compound is ethyl acetate.

14. The pharmaceutical composition according to claim 13, wherein a volume ratio of the ethyl acetate to the DMSO is 1:1 to 1:200; and
the ethyl acetate and the DMSO are separately diluted with water or mixed and then diluted with water, and then directly administered orally, injected, or sprayed to treat or prevent an animal or human disease.

15. The pharmaceutical composition according to claim 3, wherein the ketone compound is alkanone.

16. The pharmaceutical composition according to claim 3, wherein the ketone compound is acetone.

17. The pharmaceutical composition according to claim 16, wherein a volume ratio of the acetone to the DMSO is 1:1 to 1:200; and
the acetone and the DMSO are separately diluted with water or mixed and then diluted with water, and then directly administered orally, injected, or sprayed to treat or prevent an animal or human disease.

18. The pharmaceutical composition according to claim 5, wherein the alcohol compound is C₁₋₆ alkanol.

19. The pharmaceutical composition according to claim 5, wherein the alcohol compound is ethanol.

20. The pharmaceutical composition according to claim 19, wherein a volume ratio of the ethanol to the DMSO is 1:1 to 1:200; and
the ethanol and the DMSO are separately diluted with water or mixed and then diluted with water, and then directly administered orally, injected, or sprayed to treat or prevent an animal or human disease.

21. The pharmaceutical composition according to claim 13, wherein the pharmaceutical composition further comprises 4.55 v/v % lactic acid.

22. The pharmaceutical composition according to claim 16, wherein the pharmaceutical composition further comprises 4.55 v/v % lactic acid.

23. The pharmaceutical composition according to claim 19, wherein the pharmaceutical composition further comprises 4.55 v/v % lactic acid.
